(19) United States
(12) Patent Application Publication
(10) Pub. No.: US 2025/0360146 A1
(43) Pub. Date: Nov. 27, 2025

(54) COMPOSITIONS FOR SEBUM CONTROL

(71) Applicant: Dyson Operations Pte. Ltd., Singapore (SG)

(72) Inventors: Maurice Adrianus Monique VAN STEENSEL, Singapore (SG); Kai Xuan Keith TAN, Singapore (SG); Jun Xi Selwyn LOW, Singapore (SG); Eric Kian-Shiun SHIM, Singapore (SG); Liang DAI, Singapore (SG); Hwei-In Shawn LIM, Singapore (SG)

(21) Appl. No.: 18/872,390

(22) PCT Filed: Jun. 7, 2023
(86) PCT No.: PCT/IB2023/055876

§ 371 Date: Dec. 6, 2024

(30) Foreign Application Priority Data
Jun. 7, 2022 (GB) 2208320.8

(51) Int. Cl. A61K 31/56; A61K 8/63; A61K 8/9789; A61K 31/137; A61K 31/14; A61K 31/192; A61K 31/205; A61K 31/366; A61K 31/4458; A61K 31/52; A61K 31/7048; A61K 36/73; A61Q 5/00; A61Q 19/00

(52) U.S. Cl. CPC A61K 31/56; A61K 8/63; A61K 8/9789; A61K 31/137; A61K 31/14; A61K 31/192; A61K 31/205; A61K 31/366; A61K 31/4458; A61K 31/52; A61K 31/7048; A61K 36/73; A61Q 5/008; A61Q 19/008

(57) ABSTRACT

The invention provides a composition comprising oleanolic acid and the salts, prodrug and solvates thereof; and one or more compounds selected from the following group: ellagic acid, adenine, betaine, choline, miquelianin, p-coumaric acid, pipecolic acid, protocatechuic acid and tyramine, and the salts, prodrugs and solvates thereof. Such a composition is for use in reducing lipid levels in sebocytes, and the composition may find use in treating acne vulgaris and rosacea.

### FIELD OF THE INVENTION

The invention relates to compositions comprising oleanolic acid (OA) together with one or more further specified compounds, and the use of the composition in a method for decreasing lipid production in sebocytes. Also provided is the use of the composition in a cosmetic method for decreasing lipid production in the skin of an individual, and the use of the composition in a method of treating a skin disease or disorder related to increased lipid production in sebocytes.

### BACKGROUND

Sebum is an oily substance secreted by sebaceous glands in humans. Sebum is produced by sebocytes, highly-specialised epithelial cells commonly found in the skin in association with hair follicles, although there are also sebaceous glands not associated with hair follicles.

Adult human sebum typically comprises triglycerides (˜41%), wax monoesters (˜25%), free fatty acids (˜16%), and squalene (˜12%) (Cheng et al., 2004). Other components, such as keratin and cellular materials, may also be present.

Sebum forms an integral component of the epidermal barrier and the skin immune system. Sebum is a natural moisturizer for the epidermis, helping to maintain its integrity. Sebum is important in maintaining the pH of the skin surface, which may play a role in protecting the skin from exogenous (disease-causing) microbes and encouraging the growth of endogenous (resident) microflora. Sebaceous secretions in conjunction with apocrine (sweat) glands are also thought to play an important thermoregulatory role.

Sebocyte formation is controlled by multiple molecular pathways (e.g. Blimp1, Wnt, C-myc, Hedgehog) and sebum synthesis is strongly regulated by hormones, in particular by androgens such as testosterone.

Sebum is produced in a holocrine process, in which sebocytes rupture and disintegrate as they release the sebum along with cell remnants. During the terminal differentiation of sebocytes, metabolic activity is concentrated on the biosynthesis of lipids (lipogenesis), and in particular on the neosynthesis of fatty acids and squalene.

The level of sebum production varies from person to person and is influenced by sex, age, physical activity, stress, certain medications, and disease. Oily skin is commonly observed in adolescence due to hormonal changes occurring throughout puberty.

Excessive sebum production is associated with cosmetic problems, such as oily or shiny skin and poor retention of make-up, as well as medical problems. Excessive sebum production is seen in acne vulgaris, one of the most common skin diseases.

Hyperseborrhea is a scalp problem caused by excessive production of sebum. Immediate symptoms of hyperseborrhea include scalp itchiness and pain, though later symptom is hair loss. Individuals with hyperseborrhoeic skin typically exhibit sebum levels of greater than 200 μg cm⁻² measured on the forehead (as discussed, for example, in WO 2020/263188). Deregulated sebocyte differentiation also characterizes some rare benign and malignant tumors.

Cosmetic treatments for excess lipid production generally do not address the underlying causes. Rather, cosmetic treatments typically provide relief from the direct symptoms, such as oiliness, enlarged pores, acne prone skin, and irregular skin texture. For example, a common approach to treating oily or shiny skin is the use of powders that provide an immediate masking effect by absorbing the excess sebum on the skin's surface. Alternatively, astringents and cleaning agents may be used.

The known methods for reducing lipids on the skin surface are limited, producing little sustainable visible results over extended periods of time. Prolonged use of astringents and cleaning agents may exacerbate the condition.

Accordingly, there is a need to develop compositions and methods for reducing lipid production in sebocytes and in the skin of an individual.

### SUMMARY OF THE INVENTION

At its most general, the first aspect of the invention relates to a composition comprising oleanolic acid (OA) together with one or more compounds selected from the group consisting of ellagic acid, adenine, betaine, choline, miquelianin, p-coumaric acid, pipecolic acid, protocatechuic acid and tyramine, and the salts, prodrugs and solvates thereof. The inventors have found that such a composition provides superior lipid reducing effects in sebocytes.

Preferred features of the composition are set out below.

The composition may comprise two or more compounds selected from the group, such as three or more compounds, such as where at least one compound is ellagic acid.

The mole ratio of oleanolic acid to a compound selected from the group may be 1:x, where x is in the range 0.1 to 20.

The composition may comprise oleanolic acid and the salts and solvates thereof; and a plant extract comprising the one or more compounds. The plant extract may be a Fragaria vesca extract, a Rubus idaeus extract, or a Fragaria×ananassa extract, such as a Fragaria vesca extract or a Rubus idaeus extract.

The inventors have found that compositions of the first aspect reduce the production of lipids in sebocytes.

Accordingly, in a second aspect of the invention, there is provided a method for decreasing lipid production in sebocytes, the method comprising contacting the sebocytes with a composition of the first aspect. This method may be in vivo or ex vivo, such as in vitro.

The inventors have found that the compositions of the first aspect may reduce or ameliorate cosmetic problems associated with over-production of lipids in the skin, such as oily or shiny skin, oily hair, enlarged skin pores, undesirable body odour, and decreased retention of make-up products on the skin.

Accordingly, in a third aspect of the invention, there is provided a cosmetic method for decreasing lipid production in the skin of an individual, the method comprising contacting the skin with a composition of the first aspect.

The inventors have found that the compositions of the first aspect are useful in the treatment or prophylaxis of medical problems associated with over-production of lipids in the skin, such as acne vulgaris and rosacea.

Accordingly, in a fourth aspect of the invention, there is provided a composition of the first aspect for use in a method of treatment.

In a further related aspect of the invention, there is provided a composition of the first aspect for use in a method of treating a skin disease or disorder associated with over-production of lipids in the skin, such as acne vulgaris and rosacea.

These and other aspects and embodiments of the invention are described in further detail below.

### SUMMARY OF THE FIGURES

The present invention is described with reference to the figures listed below.

FIG. 1. In-vitro lipid reduction percentage of adenine, betaine, choline, ellagic acid, and miquelianin. Oleanolic acid (OA) was used at 1.25 μM whereas the other compounds were used at 10 μM. The experiment was duplicated to ensure repeatability: (top) first run; (bottom) second run.

FIG. 2. In-vitro lipid reduction percentage of p-coumaric acid, pipecolic acid, protocatechuic acid, and tyramine. Oleanolic acid (OA) was used at 1.25 μM whereas the other compounds were used at 10 μM. The experiment was duplicated to ensure repeatability: (top) first run; (bottom) second run.

FIG. 3 shows mean fluorescence intensity obtained by flow cytometry of sebocytes cells treated with AdipoRed™ dye. Cells were incubated with test compositions for 3 days. From left to right, DMSO (negative control), EGCG (positive control), oleanolic acid (OA), Rubus idaeus extract, Fragaria vesca extracts at two different concentrations, a combination of oleanolic acid and Rubus idaeus extract according to an embodiment of the invention, and two combinations of oleanolic acid and Fragaria vesca extract at two different concentrations of Fragaria vesca according to embodiments of the invention.

FIG. 4 shows mean fluorescence intensity obtained by flow cytometry of sebocytes cells treated with AdipoRed™ dye. Cells were incubated with test compositions for 3 days. From left to right, DMSO (negative control), EGCG (positive control), salicylic acid (positive control), oleanolic acid, Rubus idaeus extract, and a combination of oleanolic acid and Rubus idaeus extract according to an embodiment of the invention.

FIG. 5 shows mean fluorescence intensity obtained by flow cytometry of sebocytes cells treated with AdipoRed™ dye. Cells were incubated with test compositions for 3 days. From left to right, DMSO (negative control), EGCG (positive control), oleanolic acid, Fragaria vesca extract, and a combination of oleanolic acid and Fragaria vesca extract according to an embodiment of the invention.

Within the figures the following labels are used with respect to the comparison in a one-way analysis of variance (one-way ANOVA):
*P value: <0.05 (Statistically significant compared to DMSO)
**P value: <0.01 (Statistically significant compared to DMSO)
***P value: 0.001 (Statistically significant compared to DMSO)
****P value: <0.0001 (Statistically significant compared to DMSO)
#P value: <0.05 (Statistically significant compared to Oleanolic Acid)
##P value: <0.01 (Statistically significant compared to Oleanolic Acid)
###P value: <0.001 (Statistically significant compared to Oleanolic Acid)
####P value: <0.001 (Statistically significant compared to Oleanolic Acid)

### DETAILED DESCRIPTION OF THE INVENTION

The invention relates to compositions comprising oleanolic acid (OA) together with one or more compounds selected from the group consisting of ellagic acid, adenine, betaine, choline, miquelianin, p-coumaric acid, pipecolic acid, protocatechuic acid and tyramine, and the salts, prodrugs and solvates thereof, and the use of the composition in a method for decreasing lipid production in sebocytes. Also provided is the use of the composition in cosmetic method for decreasing lipid production in the skin of an individual, and the use of the composition in a method of treating a skin disease or disorder related to increased lipid production in sebocytes.

The following preferences may apply to all aspects of the invention as described above. The preferences may be combined in any combination.

### Oleanolic Acid

The composition of the invention contains oleanolic acid (OA) together with one or more compounds selected from the group consisting of ellagic acid, adenine, betaine, choline, miquelianin, p-coumaric acid, pipecolic acid, protocatechuic acid and tyramine, and the salts and solvates thereof.

Oleanolic acid has the CAS Registry No. [508-02-1].

A reference to oleanolic acid also includes references to the salts and solvates thereof.

The composition of the invention may comprise an effective amount, such as a therapeutically or cosmetically effective amount, of the oleanolic acid.

In some embodiments, the composition of the invention comprises the oleanolic acid in an amount of from 0.0005 wt % to 10 wt %, such as based on the total weight of the composition. In a preferred embodiment, the composition of the invention comprises oleanolic acid in an amount of from 0.1 wt % to 5 wt %, such as 0.1 wt % to 3 wt %, and such as 0.2 wt % to 3 wt %, and more preferably 0.2 wt % to 2 wt %.

The oleanolic acid may be present in an amount that is at most 2 wt %, 3 wt %, 5 wt %, or 10 wt %.

The oleanolic acid may be present in an amount that is at least 0.0005 wt %, 0.001 wt %, 0.005 wt %, 0.01 wt %, 0.05 wt %, 0.1 wt %, 0.5 wt %, or 1.0 wt %.

In one embodiment, the composition comprises a plant extract comprising the one or more compounds. Here, oleanolic acid (OA) may not be present within, or otherwise derived from, the plant extract with which it is used in combination. For example, where the plant extract is a Fragaria vesca extract or a Rubus idaeus extract, these extracts are substantially free of oleanolic acid (OA). For example, oleanolic acid may be present at less than 1 wt %, such as less than 0.1 wt %, such as less than 0.01 wt % in the plant extract.

In one embodiment, triterpenoids may not be present within, or otherwise derived from, the plant extract with which it is used in combination. For example, where the plant extract is a Fragaria vesca extract or a Rubus idaeus extract, these extracts are substantially free of triterpenoids (OA). For example, the total triterpenoids amount may be less than 1 wt %, such as less than 0.1 wt %, such as less than 0.01 wt % in the plant extract.

### Compounds

The composition of the invention comprises one or more compounds selected from the group consisting of adenine, betaine, choline, ellagic acid, miquelianin, p-coumaric acid, pipecolic acid, protocatechuic acid and tyramine.

References to each of the compounds also includes reference to the salts and solvates thereof.

The one or more compounds may be selected from the group consisting of ellagic acid, adenine, betaine, choline, miquelianin and tyramine, and the salts and solvates thereof.

The one or more compounds may be selected from the group consisting of ellagic acid, betaine, choline, miquelianin and tyramine, and the salts and solvates thereof.

The composition may comprise two or more, such as three or more, such as four or more, compounds selected from any of the groups mentioned above.

At least one of the compounds in the composition may be ellagic acid.

The composition may comprise a combination of ellagic acid, adenine, betaine, choline, miquelianin, p-coumaric acid, pipecolic acid and tyramine. Such a combination may be provided from a Rubus idaeus plant extract. Optionally the composition may additionally comprise protocatechuic acid.

The composition may comprise a combination of ellagic acid, adenine, betaine, choline, miquelianin, p-coumaric acid, and pipecolic acid. Such a combination may be provided from a Fragaria vesca plant extract. Optionally the composition may additionally comprise protocatechuic acid.
Ellagic acid has the CAS Registry No. [476-66-4].
Adenine has the CAS Registry No. [73-24-5].
Betaine has the CAS Registry No. [107-43-7].
Choline has the CAS Registry No. [62-49-7]. The choline may be provided as choline chloride.
Miquelianin has the CAS Registry No. [22688-79-5].
p-Coumaric acid has the CAS Registry No. [7400-08-0].
Pipecolic acid has the CAS Registry No. [535-75-1].
Protocatechuic acid has the CAS Registry No. [99-50-3].
Tyramine has the CAS Registry No. [051-67-2].

In some embodiments, the composition of the invention comprises a compound selected from the group in an amount of from 0.0005 wt % to 10 wt %, such as based on the total weight of the composition. In a preferred embodiment, the composition of the invention comprises a compound selected from the group in an amount of from 0.1 wt % to 5 wt %, such as 0.1 wt % to 3 wt %, and such as 0.2 wt % to 3 wt %, and more preferably 0.2 wt % to 2 wt %.

The composition may comprise a compound selected from the group may be present in an amount that is at most 2 wt %, 3 wt %, 5 wt %, or 10 wt %.

The composition may comprise a compound selected from the group may be present in an amount that is at least 0.0005 wt %, 0.001 wt %, 0.005 wt %, 0.01 wt %, 0.05 wt %, 0.1 wt %, 0.5 wt %, or 1.0 wt %.

In one embodiment, each compound that is selected from the group is present in an amount as specified above.

In the composition of the invention, the mole ratio of oleanolic acid to a compound selected from the group is 1:x, where x is in the range 0.1 to 20. The mole ratio of oleanolic acid to a compound selected from the group may be 1:x, where x is in the range 1 to 10, such as 2 to 10, such as 5 to 10, such as about 8.

In one embodiment, each compound that is selected from the group may be present in an amount relative to oleanolic acid as specified above.

### Salts and Solvates

The oleanolic acid (OA) may be provided in free base form. Alternatively, the oleanolic acid (OA) may be provided in the form of a salt, such as a pharmaceutically or cosmetically acceptable salt.

Examples of pharmaceutically acceptable salts are discussed in Berge et al., 1977, “Pharmaceutically Acceptable Salts,” J. Pharm. Sci., Vol. 66, pp. 1-19.

In some embodiments, a compound, such as oleanolic acid, is provided in anionic form, such as a deprotonated form, together with a suitable counter cation.

Suitable counter cations include both organic and inorganic cations. Suitable counterions include both inorganic and organic cations. Examples of suitable inorganic cations include alkali metal ions such as Na⁺ and K⁺, alkaline earth cations such as Ca²⁺ and Mg²⁺, and other cations such as Al³⁺. Examples of suitable organic cations include the ammonium ion (i.e., NH₄⁺) and substituted ammonium ions (e.g., NH₃R⁺, NH₂R₂⁺, NHR₃⁺, NR₄⁺). Examples of substituted ammonium ions include those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

In some embodiments, a compound, such as choline, is provided in cationic form, together with a suitable counter cation.

Unless otherwise specified, a reference to a particular compound also includes salt forms thereof.

A compound in the composition, may be provided in the form of a solvate (a complex of solute (e.g., compound, salt of compound) and solvent). Examples of solvates include hydrates, for example, a mono-hydrate, a di-hydrate and a tri-hydrate.

A compound in the composition may be provided in desolvated form, for example, in dehydrated form.

### Plant Extracts

The composition of the invention comprises one or more compounds selected from the group consisting of ellagic acid, adenine, betaine, choline, miquelianin, p-coumaric acid, pipecolic acid, protocatechuic acid and tyramine.

The one or more compounds may be derived from or derivable from a plant extract.

In one embodiment, the composition of the invention comprises:
oleanolic acid and the salts and solvates thereof; and
a plant extract comprising the one or more compounds.

Thus, the composition of the invention may comprise a plant extract.

The plant extract may be selected from extracts of plants belonging to the genus Fragaria and Rubus.

Suitable plant extracts are commercially available and can be purchased from, for example, BOC Sciences and BOCSCI (New York, USA).

Suitable species of plants belonging to the genus Rubus include Rubus idaeus (red raspberry). Rubus idaeus is widely distributed throughout Europe and is native to, for example, the United Kingdom.

Suitable species of plants belonging to the genus Fragaria include Fragaria vesca (wild strawberry) and Fragaria×ananassa (garden strawberry). Fragaria vesca is widely distributed throughout Europe and is native to, for example, the United Kingdom. Fragaria×ananassa is widely cultivated across Europe, including the United Kingdom.

The plant extract may be derived from any suitable part of the plant. Suitable parts of the plant include roots, stems, leaves, flowers, fruits and seeds.

Examples of suitable fruit extracts include Fragaria vesca, Rubus idaeus and Fragaria×ananassa fruit extracts.

Typically, the plant extract is an aqueous extract of the plant or plant part. The aqueous extract may be used as the dried aqueous extract of the plant or plant part.

In a preferred embodiment, the plant extract is selected from Fragaria vesca fruit extract and Rubus idaeus fruit extract.

In a preferred embodiment, the plant extract is Rubus idaeus extract.

In a further embodiment, the plant extract is Rubus idaeus fruit extract.

In a preferred embodiment, the plant extract is Fragaria vesca extract.

In a further embodiment, the plant extract is Fragaria vesca fruit extract.

In a preferred embodiment, the plant extract is Fragaria×ananassa extract.

In a further embodiment, the plant extract is Fragaria×ananassa fruit extract.

As noted above, oleanolic acid may be substantially absent from, such as may not be present within, or otherwise derived from, the plant extract with which it is used in combination.

The composition of the invention may comprise an effective amount, such as a therapeutically or cosmetically effective amount, of the plant extract described herein.

The plant extract may be provided at sufficient amount to give the one or more compounds at an amount such as described above, such as at an effective amount, such as a therapeutically or cosmetically effective amount.

A Rubus idaeus extract may comprise one or more of, such as all of, the compounds given in Table 1 (see the Examples section), at an amount in the range given. The extract here may be a dry extract.

A Rubus idaeus extract may be obtained or obtainable from raspberry fruit. The extract may be obtained by extraction with water, concentrated and spray dried.

A Fragaria vesca extract may comprise one or more of, such as all of, the following compounds given in Table 2 (see the Examples section), at an amount in the range given. The extract here may be a dry extract.

A Fragaria vesca extract may be obtained or obtainable from strawberry fruit. The extract may be obtained by extraction with water, concentrated and spray dried.

In some embodiments, the composition of the invention comprises the plant extract in an amount of from 0.005 wt % to 50 wt %, such as based on the total weight of the composition. In a preferred embodiment, the composition of the invention comprises the plant extract in an amount of from 0.005 wt % to 30 wt %, such as 0.05 wt % to 10 wt %, more preferably 0.1 wt % to 3 wt %.

The plant extract may be present in an amount that is at most 2 wt %, 3 wt %, 5 wt %, 10 wt %, 15 wt %, 20 wt %, 25 wt %, 30 wt %, 35 wt %, 45 wt %, or 50 wt %.

The plant extract may be present in an amount that is at least 0.005 wt %, 0.01 wt %, 0.05 wt %, 0.1 wt %, 0.5 wt %, or 1 wt %.

### Compositions

The compositions of the invention may be formulated for cosmetic or therapeutic uses. Accordingly, the compositions of the invention may additionally comprise one or more pharmaceutically or cosmetically ingredients.

Pharmaceutically cosmetically acceptable ingredients are those which, within the scope of sound judgment, are suitable for use in contact with the tissues of the subject in question (e.g., human) without excessive toxicity, irritation, allergic response, or other problem or complication, commensurate with a reasonable benefit/risk ratio. Each ingredient must also be compatible with the other ingredients of the composition.

In one embodiment, the composition comprises one or more ingredients selected from solvents, oils, surfactants, thickeners, humectants, and preservatives.

Examples of suitable solvents includes water; mono-alcohols such as ethanol, isopropanol, benzyl alcohol, and phenylethyl alcohol; polyalcohols such as ethylene glycol, propylene glycol, butane-1,3-diol, mannitol, sorbitol, glycerol, and erythritol; and glycol ethers such as ethylene glycol monomethyl ether and diethylene glycol monomethyl ether.

Examples of suitable oils include mineral oils, plant oils and waxes.

Examples of suitable plant oils include algal oil, annatto oil, argan oil, almond oil, apricot kernel oil, avocado oil, babassu oil, Brazil nut butter, butter, cashew butter, castor oil, camellia oil, cheery kernel oil, cocoa butter, coconut oil, corn oil, cottonseed oil, fish oil, grape seed oil, gardenia oil, ghee, hazelnut oil, jatropha oil, jojoba oil, kokum oil, linseed oil, macadamia oil, maize oil, mango seed oil, mango butter, mineral oil, mink oil, olive oil, palm oil, palm kernel oil, peach kernel 5 oil, peanut butter, peanut oil, plum kernel oil, pomegranate oil, rapeseed seed oil, rice bran oil, rosehip oil, sal oil, sesame oil, shea butter, soybean oil, squalene, sunflower oil, teas seed oil, walnut oil.

Examples of suitable waxes include bayberry wax, beeswax, camauba wax (palm wax), candelilla wax, ceresin, jojoba butter, lanolin wax, montan wax, ozokerite, polyglyceryl-3-beeswax, polyglyceryl-8-pentastearate, Japan wax, microcrystalline wax, paraffin wax, isoparaffin, vaseline solid paraffin, squalene, oligomer olefins, synthetic candelilla wax, synthetic camauba, synthetic beeswax

Surfactants (surface-active agents) may act as dispersants or wetting agents. Examples of suitable surfactants include anionic surfactants, cationic surfactants, non-ionic surfactants, and amphoteric (zwitterionic) surfactants.

Examples of suitable anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate

Examples of suitable cationic surfactants include behentrimonium chloride, cocotrimonium chloride, cethethyldimonium bromide, dibehenyidimonium chloride, dihydrogenated tallow benzylmonium chloride, disoyadimonium chloride, ditallowdimonium chloride, hydroxycetyl hydroxyethyl dimonium chloride, hydroxyethyl behenamidopropyl dimonium chloride, Hydroxyethyl cetyidimonium chloride, hydroxyethyl tallowdimonium chloride, myristalkonium chloride, PEG-2 oleamonium chloride, PEG-5 stearmonium chloride, PEG-15 cocoyl quaternium 4, PEG-2 stearalkonium 4, lauryltrimonium chloride; Quatemium-16; Quatemium-18, lauralkonium chloride, olealkmonium chloride, cetylpyridinium chloride, Polyquaternium-5, Polyquatemium-6, Polyquatemium-7, Polyquatemium-10, Polyquaternium-22, Polyquatemium-37, Polyquatemium-39, Polyquatemium-47, cetyl trimonium chloride, dilauryidimonium chloride, cetalkonium chloride, dicetyidimonium chloride, soyatrimonium chloride, and stearyl octyl dimonium methosulfate.

Examples of suitable non-ionic surfactants include fatty alcohol ethoxylates such as octaethylene glycol monododecyl ether, and pentaethylene glycol monododecyl ether, alkylphenol ethoxylates (APEs or APEOs) such as Nonoxynol-4, Nonoxynol-7, Nonoxynol-9, Nonoxynol-14, Nonoxynol-15, Nonoxynol-18, and triton X-100; glycerol fatty acid esters such as glycerol monostearate, and glycerol monolaurate; sorbitol fatty acid esters such as sorbitan monolaurate, sorbitan monostearate, sorbitan tristearate, polysorbate (Tween) 20, polysorbate 40, polysorbate 60, and polysorbate 80.

Examples of suitable amphoteric (zwitterionic) surfactants include cocamidopropyl hydroxysultaine, cocamidopropyl betaine, lauryl betaine, lauryldimethylamine oxide, and myristamine oxide.

Examples of suitable thickeners (rheological modifiers) include gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar, pectin; gelatin; starch and its derivatives; chitosan and its derivatives such as hydroxyethyl chitosan; synthetic polymers such as polyvinyl alcohol, PVM/MA copolymer, PVM/MA decadiene crosspolymer, poly(ethylene oxide) based thickeners;

Humectants may act as hygroscopic agents, increasing the amount of water absorbed or retained by the composition. Examples of suitable humectants include acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, and sodium PCA.

Examples of suitable preservatives includes methyl p-hydroxybenzoate, propyl p-hydroxybenzoate and sorbic acid. Methyldibromo glutaronitrile (MDBGN), benzyl alcohol, imidazolidinyl urea 1,3-bis(hydroxymethyl)-5,5-dimethyl-2,3-imidazolidinedione (DMDM hydantoin), methylchloroisothiazolinone and methylisothiazolinone, phenoxyethanol, and sodium benzoate.

In one embodiment, the composition comprises one or more ingredients selected from emollients, anti-inflammatory agents, antioxidants and UV blocking agents.

Examples of suitable emollients include fatty esters such as isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C12 to C16 fatty alcohol lactate, isopropyl lanolate, and 2-ethyl-hexyl salicylate.

Examples of suitable anti-inflammatory ingredients include cyclo-oxygenase (e.g., COX-1 or COX-2) or Lipoxygenase (e.g., LOX-5) enzyme inhibitors such as ascorbic acid, ascorbic acid derivatives, vitamin E, vitamin E derivatives, tocotrienol, rutin, quercetin, hesperidin (Citrus sinensis), hesperetin (Citrus sinensis), diosmin (Citrus sinensis), mangiferin (Mangifera indica), mangostin (Garcinia mangostana), cyanidin (Vaccinium myrtillus), astaxanthin (Haematococcus algae), lutein (Tagetes patula), lycopene (Lycopersicum esculentum), resveratrol (Polygonum cuspidatum), tetrahydrocurcumin (Curcuma longa), rosmarinic acid (Rosmarinus officinalis), hypericin (Hypericum perforatum), ellagic acid (Punica granatum), chlorogenic acid (Vaccinium vulgars), oleuropein (Olea europaea), alpha-lipoic acid, glutathione, andrographolide (Andrographis paniculata), grapeseed extract, green tea extract, polyphenols, pycnogenol (pine bark extract), white tea extract, black tea extract, (Andrographis paniculata), camosine, and niacinamide. Further examples of suitable anti-inflammatory composition can additionally be selected from horse chestnut extract (Aesculus hippocastanum extract), esculin, escin, yohimbine, Capsicum oleoresin, capsaicin, niacin, niacin esters, methyl nicotinate, benzyl nicotinate, ruscogenins (Butchers Broom extract; Ruscus aculeatus extract), diosgenin (Trigonel afoenumgraecum, fenugreek), emblica extract (Phyllanthus emblica extract), asiaticoside (Centella asiatica extract), Boswellia extract (Boswellia serrata), sericoside, visnadine, thiocolchicoside, grapeseed extract, ginger root extract (Zingiber offcianale), piperine, vitamin K, melilot (Melilotus officinalis extract), glycyrrhetinic acid, ursolic acid, sericoside (Terminalia sericea extract), darutoside (Siegesbeckia orientalis extract), Amni visnaga extract, vine leaf extract (Vitis vinifera), apigenin, phytosan, luteolin.

Examples of suitable antioxidant ingredients include ascorbic acid, ascorbic acid derivatives glucosamine ascorbate, arginine ascorbate, lysine ascorbate, glutathione ascorbate, nicotinamide ascorbate, niacin ascorbate, allantoin ascorbate, creatine ascorbate, creatinine ascorbate, chondroitin ascorbate, chitosan ascorbate, camosine ascorbate, vitamin E, vitamin E derivatives, tocotrienol, rutin, quercetin, hesperidin (Citrus sinensis), hesperetin (Citrus sinensis), diosmin (Citrus sinensis), mangiferin (Mangifera indica), mangostin (Garcinia mangostana), cyanidin (Vaccinium myrtillus), astaxanthin (Haematococcus algae), lutein (Tagetes patula), lycopene (Lycopersicum esculentum), resveratrol (Polygonum cuspidatum), tetrahydrocurcumin (Curcuma longa), rosmarinic acid (Rosmarinus officinalis), hypericin (Hypericum perforatum), ellagic acid (Punica granatum), chlorogenic acid (Vaccinium vulgaris), oleuropein (Olea europaea), alpha-lipoic acid, niacinamide lipoate, glutathione, andrographolide (Andrographis paniculata), camosine, niacinamide, Potentilla erecta extract, polyphenols, grapeseed extract, pycnogenol (pine bark extract), pyridoxine, magnolol, honokiol, paeonol, resacetophenone, quinacetophenone, arbutin and kojic acid.

Examples of suitable UV blocking agents (sunscreen active agents) include octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl PABA, diethanolamine p-methoxy cinnamate, ethyl 4-bis(hydroxypropyl) aminobenzoate, 2-ethylhexyl-2-cyano-3,3-diphenylacrylate, homomenthyl salicylate, glyceryl aminobenzoate, dihydroxyacetone, octyl dimethyl PABA, 2-phenylbenzimidazole-5-sulfonic acid, triethanolamine salicylate, zinc oxide, titanium oxide, and mixtures thereof.

Further components suitable for use in the composition include fragrances, pH adjusters, pigments, odour absorbers, antimicrobial agents, antifungal agents, chelating agents, and saccharides.

The compositions of the present invention are typically formulated for topical use.

The composition of the present invention may be formulated as a solution, liquid, lotion, cream, emulsion, dispersion, gel, or paste. Examples of suitable emulsions include two-phase emulsions comprising an aqueous phase and an oil phase such as oil-in-water (O/W) and water-in-oil (W/O), as well as complex emulsions such as triple emulsions (O/W/O and W/O/W).

The composition and formulations may be prepared by any methods well known in the art. Such methods include the step of bringing into association oleanolic acid and/or the one or more compounds, which one or more compounds may be present in a plant extract, with a carrier which constitutes one or more accessory ingredients. In general, the formulations are prepared by uniformly mixing oleanolic acid and/or the one or more compounds, which one or more compounds may be present in a plant fungal extract, with a carrier (e.g., a liquid carrier, a finely divided solid carrier, etc.).

The compositions of the present invention can be formulated as both cosmetic and pharmaceutical products.

In one embodiment, there is provided a personal care product comprising a composition of the invention. Suitable personal care products include skin care products, hair care products, cleansing products, and cosmetic powders and liquids.

Examples of suitable skin care products include skin/hand lotions, skin/hand creams, skin/hand ointments, skin/hand pastes, skin toner, shaving gels, shaving creams, sunscreens, deodorants, antiperspirants, suntan lotions, after sun, aftershaves, body oils, bath oils, and bubble baths. Examples of suitable hair care products include conditioners, hair detangling lotion, styling gel, styling creams, styling waxes, styling lotions, mousses, spray gels, hair tonics, spritzes, and pomades. Examples of suitable cleansing products include liquid soaps, bar soaps, body washes, skin cleansers, and shampoos. Examples of suitable cosmetic powders and liquids include blusher, body powder, bronzing powder, eye shadow, foundation, face powder, lip powder, powder makeup, liquid bronzer, eyeliner, lip gloss, lipstick, and mascara.

### Method and Uses for Decreasing Lipid Production

The compositions of the invention reduce the production of lipids in sebocytes.

The invention also provides a method for decreasing lipid production in sebocytes, the method comprising contacting the sebocytes with a composition of the invention.

Preferred features of the composition, such as the oleanolic acid, the one or more compounds and the plant extract comprising the one or more compounds, are set out above.

In one embodiment, the method for decreasing lipid production in sebocytes is in vivo. In one embodiment, the method for decreasing lipid production in sebocytes ex vivo, such as in vitro.

In one embodiment, the method for decreasing lipid production in sebocytes comprises contacting the sebocytes with an effective amount of a composition of the invention. An effective amount of the composition provides a detectable reduction in the production of lipids in the sebocytes. Methods for detecting and quantifying the production of lipids in sebocytes include, for example, a fluorescence assay using a suitable lipid-detecting dye, such as AdipoRed™.

In one embodiment, the method for decreasing lipid production in sebocytes comprises contacting the sebocytes with an amount of a composition of the invention suitable for reducing the production of lipids by 10% or more, preferably 15% or more, more preferably 20% or more, even more preferably 20% or more, and most preferably 30% or more.

In one embodiment, the method for decreasing lipid production in sebocytes decreases lipid production by 5% or more, preferably 10% or more, preferably 15% or more, more preferably 20% or more, even more preferably 20% or more, and most preferably 30% or more.

The invention also provides a composition of the invention for use in a method of decreasing lipid production in sebocytes.

Preferred features of the composition are set out above. Preferred features of the method for decreasing lipid production in sebocytes are also set out above.

The invention also provides the use of a composition of the invention to decrease lipid production in sebocytes.

Preferred features of the composition, such as the oleanolic acid the one or more compounds, and the plant extract comprising the one or more compounds, are set out above. Preferred features of the method for decreasing lipid production in sebocytes also apply to the use of the composition to decrease lipid production in sebocytes.

### Cosmetic Methods and Uses

The compositions of the invention reduce lipid production in sebocytes, such as those in the skin of an individual. As such, the compositions of the invention can reduce or ameliorate cosmetic problems associated with over-production of lipids in the skin. Cosmetic problems associated with overproduction of lipids in the skin include oily or shiny skin, oily hair, enlarged skin pores, undesirable body odour, and decreased retention of make-up products on the skin.

Accordingly, the invention provides a cosmetic method for decreasing lipid production in the skin of an individual, the method comprising contacting the skin with a composition of the invention.

Preferred features of the composition are set out above.

In one embodiment, the cosmetic method for decreasing lipid production in the skin of an individual is not a method of treatment. In one embodiment, the cosmetic method for decreasing lipid production in the skin of an individual is not a method of treatment of the human or animal body by therapy.

In one embodiment, the cosmetic method reduces lipid production in sebocytes in the skin of an individual.

In one embodiment, the skin is skin on the head, such as on the face, mouth, neck, or scalp. In one embodiment, the skin is skin on the chest, back, arms, legs, or hands.

The benefits of decreasing lipid production in the skin of an individual include reducing the oily appearance of the skin, controlling surface oil, minimizing skin pores and reducing undesirable body odour. In one embodiment, the cosmetic method decreases lipid production in the skin, thereby achieving an effect selected from reducing the oily appearance of the skin, controlling skin surface oil, minimizing skin pores and reducing undesirable body odour.

In one embodiment, the individual is in need of cosmetic treatment. Individuals in need of cosmetic treatment may have a condition associated with overproduction of lipids in the skin, such as oily or shiny skin, oily hair, enlarged skin pores, and undesirable body odour. In one embodiment, the individual has a cosmetic condition selected from oily or shiny skin, oily hair, enlarged skin pores, and undesirable body odour.

The invention also provides a composition of the invention for use in a cosmetic method of decreasing lipid production in the skin of an individual.

Preferred features of the composition are set out above. Preferred features of the cosmetic method for decreasing lipid production in the skin of an individual are also set out above.

The invention also provides the use of a composition of the invention to decrease lipid production in the skin of an individual.

Preferred features of the composition are set out above. Preferred features of the cosmetic method for decreasing lipid production in the skin of an individual also apply to the use of the composition to decrease lipid production in the skin of an individual.

### Medical Methods and Uses

The compositions of the invention reduce lipid production in sebocytes, such as those in the skin of an individual. As such, the compositions of the invention are useful in the treatment or prophylaxis (prevention) of medical problems associated with over-production of lipids in the skin. Medical problems associated with overproduction of lipids in the skin include acne vulgaris and rosacea.

Accordingly, the invention provides a composition of the invention for use in a method of treatment, such as a method of treatment of the human or animal body by therapy.

In one embodiment, the method of treatment is a method of treatment of a disorder (e.g. a disease) associated with over-production of lipids in the skin. In one embodiment, the method of treatment is a method of treatment of a disorder associated with over-production of lipids in sebocytes.

In one embodiment, the skin is skin on the head, such as on the face, mouth, neck, or scalp. In one embodiment, the skin is skin on the chest, back, arms, legs, or hands.

In one embodiment, the treatment is treatment of acne vulgaris. In one embodiment, the treatment is treatment of rosacea.

In one embodiment, the treatment is administered to a subject in need of treatment.

The subject in need of treatment (the patient) may be a mammal such as a human.

The subject in need of treatment may be an adult or juvenile.

In a preferred embodiment, the subject in need of treatment is a human, more preferably an adult human.

Alternatively, the subject in need of treatment is a non-human animal used in laboratory research.

In one embodiment, the treatment is administered by any convenient route of administration. In a preferred embodiment, the treatment is administered topically (i.e. at the site of desired action).

In one embodiment, the treatment comprises administering a therapeutically-effective amount of the composition to a subject in need of treatment.

It will be appreciated by one of skill in the art that appropriate dosages of the compositions described herein, can vary from patient to patient. Determining the optimal dosage will generally involve the balancing of the level of therapeutic benefit against any risk or deleterious side effects. The selected dosage level will depend on a variety of factors including, but not limited to, the activity of the oleanolic acid, the one or more compounds, or the plant extract comprising the one or more compounds, the route of administration, the time of administration, the rate of excretion of the oleanolic acid, the one or more compounds, or the plant extract comprising the one or more compounds, the duration of the treatment, other drugs, compounds, and/or materials used in combination, the severity of the disorder, and the species, sex, age, weight, condition, general health, and prior medical history of the patient. The amount of oleanolic acid and the one or more compounds, or the plant extract comprising the one or more compounds, and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose (application), continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

### Other Preferences

Each and every compatible combination of the embodiments described above is explicitly disclosed herein, as if each and every combination was individually and explicitly recited.

Various further aspects and embodiments of the invention will be apparent to those skilled in the art in view of the present disclosure.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### Examples

Certain aspects and embodiments of the invention will now be illustrated by way of example and with reference to the figures described above.

### Materials

Dimethyl sulfoxide (DMSO) epigallocatechin gallate (EGCG), and salicylic acid (SA) were purchased from Sigma-Aldrich.

Oleanolic acid (OA) was purchased from Sabinsa Corporation.

Adenine, betaine, choline, ellagic acid, miquelianin, p-coumaric acid, pipecolic acid, protocatechuic acid or tyramine were purchased from Sigma-Aldrich Pte Ltd at Reagent grade.

Fragaria vesca (FV) plant extracts were purchased from BOC Sciences.

Hedera helix extract (HH), Garcinia mangostana (GM), Ilex aquifolium extract (IA) and

Rubus idaeus extract (RI) extract were purchased from BOCSCI (New York, USA).

Rubus idaeus (RI) plant extracts were purchased from BOC Sciences or BOCSCI (New York, USA).

The plant extracts may be obtained as described below.

| Extract | Extraction Method |
|---|---|
| Hedera helix extract (HH) | From ivy; Extracted by water, concentrated |
| | and dried |
| Ilex aquifolium extract (IA) | From holly; Extracted by water, concentrated |
| | and dried |
| Rubus idaeus extract (RI) | From raspberry fruit; Extracted by water, |
| | concentrated and spray dried |
| Fragaria vesca extract (FV) | From strawberry fruit; Extracted by water, |
| | concentrated and spray dried |

Primary sebocytes were derived from the forehead of a 48-year-old Chinese male. The same cell line was used in all experiments.

Rubus idaeus Extract

Using suitable analytical techniques, three Rubus idaeus solid plant extracts were deconvoluted and the following compounds were identified in the extract. The preparation of the liquid sample for analysis from the solid extract is described below.

TABLE 1
Components of Rubus idaeus solid plant extract

| Compound | Concentration Range (μg/g = ppm) |
|---|---|
| Adenine | 0.7-7.1 |
| Betaine | 1.2-3.4 |
| Choline | 5.4-18.4 |
| Ellagic acid | 21.9-439.8 |
| Miquelianin | 0.2-21.4 |
| p-coumaric acid | 0.2-9.2 |
| Pipecolic acid | 1.0-74.4 |
| Tyramine | 17-182.9 |

Fragaria vesca Extract

Using suitable analytical techniques, a Fragaria vesca solid plant extract was deconvoluted and the following compounds were identified in the extract. The preparation of the liquid sample for analysis from the solid extract is described below.

TABLE 2
Components of Fragaria vesca solid plant extract

| Compound | Concentration Range (μg/g = ppm) |
|---|---|
| Adenine | 206 |
| Betaine | 238 |
| Choline | 261 |
| Ellagic acid | 346 |
| p-Coumaric acid | 140 |
| Pipecolic acid | 244 |
| Miquelianin | 306 |

### Analytical Methods

Liquid samples were prepared for analysis following the protocol set out below:
1. 100 mg of dry powder of a Rubus idaeus or a Fragaria vesca extract sample was weighted and dissolved in 1 mL of 50% ACN/water solution, then diluted for 10× for LC-MS analysis;
2. Standard compounds (adenine, betaine, choline, ellagic acid, miquelianin, p-coumaric acid, pipecolic acid, protocatechuic acid or tyramine) were dissolved in 50% ACN/water solution in 2 mL tubes, vortexed and sonicated for 10 minutes. A standard mixture with a final concentration of 1 ppm was prepared by pooling the individual standard adding 10% (VN) of known-concentration internal standard (ISTD); and
3. 15 μL of the samples and standard prepared were injected into LC-MS.

### Liquid Chromatography

The analyses were conducted using a Waters Xevo@ G2-S QToF with UltraPerformance LC@ with an HSS T3 1.8 um column and detection parameters described below:
1. The mobile phase consisted of (A) Water+0.1% formic acid and (B) Acetonitrile+0.1% formic acid;
2. The gradient was 0-1 min, 1% B isocratic; 1-11 min, 1-40% B linear; 11-13 min. 40-70% B linear; 13-15 min, 70-99% B linear; 15-16 min, 99% B isocratic; and 16-17 min, 99-1% B linear; followed by 3 min of re-equilibration of the column with 1% B isocratic before the next run; and
3. A constant 40° C. column and 4° C. auto-sampler temperatures were kept throughout the 20-minute analysis. The injection volume of the sample is 15 μL with a 2 μL sample loop.

### Mass Spectrometry

The mass spectrum data were acquired at ESI positive ion mode and MS centroid acquisition mode with the following acquisition parameters to obtain the product ion spectra:

| Parameter | Positive Mode |
|---|---|
| Capillary voltage (V) | 1,500 |
| Source temperature (° C.) | 120 |
| Desolvation temperature (° C.) | 600 |
| Cone gas flow (l/h) | 150 |
| Desolvation gas flow (l/h) | 1000 |
| Mass range (Da) | 50-1200 |
| Scan time (s) | 0.10 |

The amount of the nominated compounds was then quantified by comparing the retention time and mass-to-charge ratio with the standard compounds.

### Characterisation Methods

Flow cytometry experiments were conducted on the BD LSR Fortessa X-20 as follows: Sebocytes stained with a lipid dye (AdipoRed™) were first analyzed according to size and granularity with only single cells selected for AdipoRed™ signal intensity measurement. The AdipoRed™ signal was measured using the 488 nm FITC blue laser and the value for the average fluorescence intensity of 10,000 cells obtained. The average fluorescence intensity is a measure of the average lipid quantity in 10,000 sebocytes. Cells treated with compositions of the invention were compared to vehicle-treated cells to determine the percentage of lipid reduction.

### Primary Sebocytes Culture

Primary sebocytes were cultured to 80% confluency in Complete Culture Medium with Y-27632 (CCMY) consisting of 3:1 DMEM (Gibco, 11995-065)/F12 (Gibco, 31765-035), supplemented with 10% fetal bovine serum (Hyclone, SV30160.03), 1× Penstrep (Gibco, 15140-122), 0.2 μg/mL Epidermal Growth Factor (PeproTech, AF-100-15-1MG), 1 μg/mL Hydrocortisone (Sigma-Aldrich, H0888), 10⁻⁹ M cholera toxin (Sigma-Aldrich, C8052-2MG) and 10 μM Y-27632 (Tocris, 1254/10) at 37° C. with 5% CO₂. The cells were washed with PBS buffer [5 mL for a 10 cm culture plate] and then incubated at 37° C. with 0.125% Trypsin-EDTA (Gibco, 15400054) [2 mL of Trypsin for a 10 cm culture plate] for 5-10 minutes. When all the cells had detached, neutralisation media consisting of DMEM with 10% FBS and 1× Penstrep (2 mL of neutralisation media for a 10 cm culture plate) was added, and the cells were transferred to a 15 mL conical tube and centrifuged at 1,000 rpm for 5 minutes.

After centrifugation, a cell pellet was formed, the supernatant was discarded and the cells were resuspended in culture medium.

The cells were cultured in a CelCulture CO₂ Incubator 170L (Esco Lifesciences).

Cells were counted by loading onto a hemocytometer, with the cells observed using a CKX41 Inverted Microscope (Olympus) and manually counted.

### General Test Protocol

A test composition was added to the well of a 24 well plate to achieve a final concentration of 1.25 μM for Oleanolic acid (OA), 50 μM for EGCG, 10 μM for the other compounds, and 31.25 μg/mL for the extract (4 μL of a 3,125 μg/mL stock solution made up in DMSO into 396 μL of culture media). In some experiments, where stated, the plant extract was used at 62.5 μg/mL.

Primary sebocytes were seeded at 60×10⁶ cells per well (396 μL) in culture media. The cells were incubated for 3 days at 37° C. and 5% CO₂. The media was discarded, AdipoRed™ dye (0.06% v/v in PBS) was added and the cells incubated at 37° C. for 20 minutes. The staining solution was discarded, trypsin (0.125%; 250 μL) was added and the cells incubated at 37° C. for 5-10 minutes. When all cells had detached, fixative solution (250 μL; MEM without phenol red, 10% fetal bovine serum and 4% paraformaldehyde) was added and the cells transferred to 5 mL polystyrene tubes. The tubes were stored on ice before flow cytometry. Mean fluorescence intensity of the lipid signal can be obtained from the flow cytometer plot which are used to determine the inhibitory effect of the compounds on the sebocytes.

### Sebum Reduction Examples

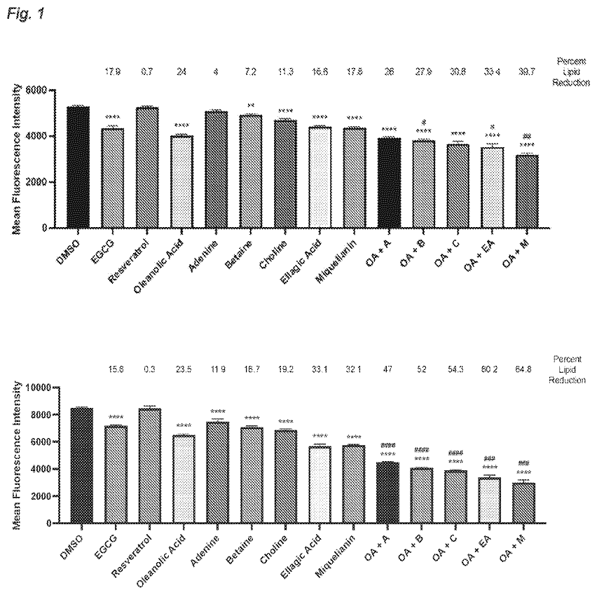

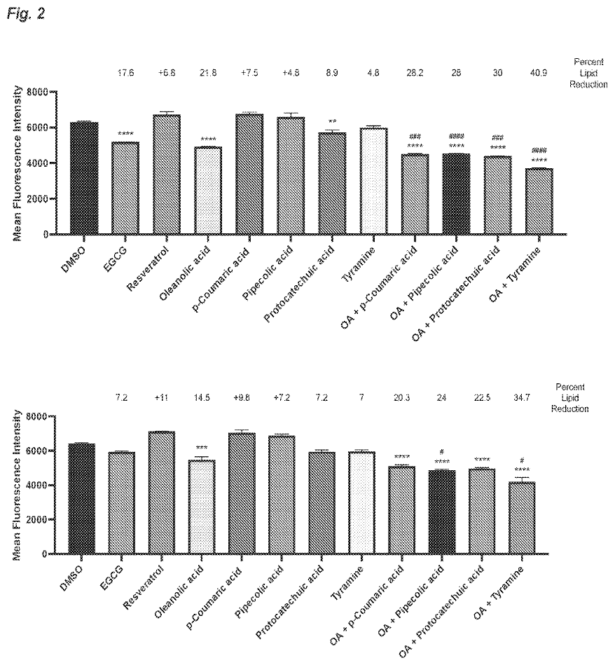

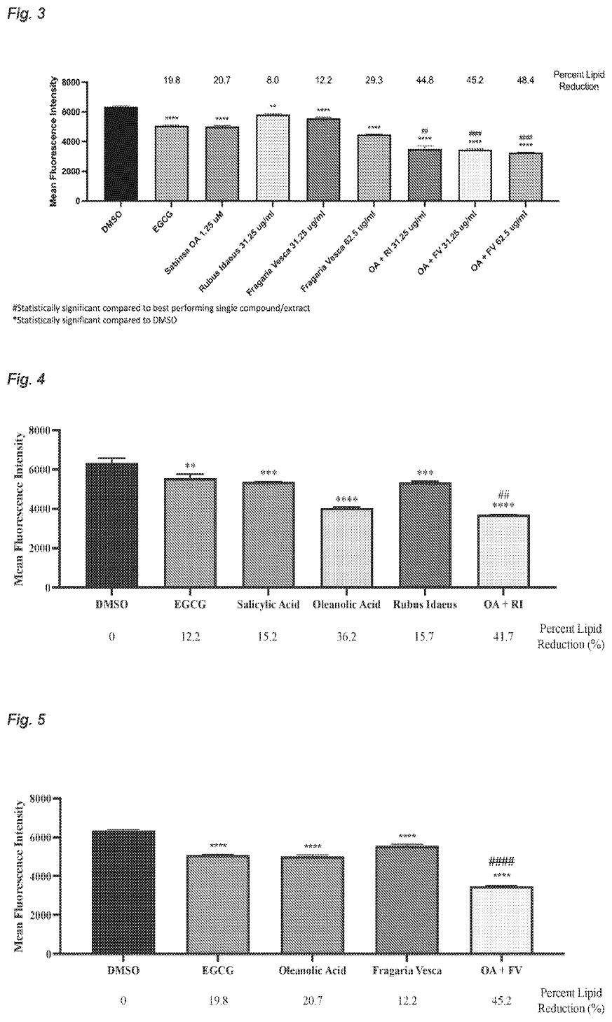

The lipid reduction effects of the compositions of the invention were measured against DMSO as a negative control, and against the known lipid-reducing compound epigallocatechin gallate (EGCG) as a positive control. The results are shown in Tables 3 and 4, and FIGS. 1 to 3.

TABLE 3
In-vitro lipid reduction percentages of the entries.

| | | Percentage Reduction in Mean |
|---|---|---|
| Entry | Composition | Fluorescence Intensity vs DMSO |
| 1 | DMSO (vehicle) | 00.0 |
| 2 | EGCG | 16.8 ± 01.5 |
| 3 | Oleanolic Acid (OA) | 23.8 ± 00.3 |
| 4 | adenine | 8.0 ± 04.0 |
| 5 | betaine | 12.0 ± 04.8 |
| 6 | choline | 15.3 ± 04.0 |
| 7 | ellagic acid | 24.9 ± 08.3 |
| 8 | miquelianin | 25.0 ± 07.1 |
| 9 | OA + adenine | 36.5 ± 10.5 |
| 10 | OA + betaine | 40.0 ± 12.1 |
| 11 | OA + choline | 42.6 ± 11.8 |
| 12 | OA + ellagic acid | 46.8 ± 13.4 |
| 13 | OA + miquelianin | 52.3 ± 12.6 |
| 14* | EGCG | 12.4 ± 5.2 |
| 15* | Oleanolic Acid (OA) | 18.2 ± 3.7 |
| 16* | p-coumaric acid | −8.7 ± 1.2 |
| 17* | pipecolic acid | −6.0 ± 1.2 |
| 18* | protocatechuic acid | 8.1 ± 0.8 |
| 19* | tyramine | 5.9 ± 1.1 |
| 20* | OA + p-coumaric acid | 24.3 ± 4.0 |
| 21* | OA + pipecolic acid | 26.0 ± 2.0 |
| 22* | OA + protocatechuic acid | 26.3 ± 3.8 |
| 23* | OA + tyramine | 37.8 ± 3.1 |

Note:
OA was used at 1.25 μM, EGCG at 50 μM, whereas the other compounds were used at 10 μM.
*Data measured in a different set of experiments

A further series of experiments was subsequently performed testing Oleanolic Acid alone and together with Rubus idaeus and Fragaria vesca plant extracts comprising the components as set out in Tables 1 and 2 respectively, and the results are set out below. The experiments were undertaken as before, with the concentrations of the extracts and compounds as noted.

TABLE 4
In-vitro lipid reduction percentages of the entries.

| | | Percentage Reduction in Mean |
|---|---|---|
| Entry | Composition | Fluorescence Intensity vs DMSO |
| 1 | DMSO (vehicle) | 00.0 |
| 2 | EGCG (50 μM) | 19.8 |
| 3 | Oleanolic Acid (1.25 μM) | 20.7 |
| 4 | Rubus idaeus (31.25 μg/mL) | 8.0 |
| 5 | Fragaria vesca (31.25 μg/mL) | 12.2 |
| 6 | Fragaria vesca (62.5 μg/mL) | 29.3 |
| 7 | OA + RI (31.25 μg/mL) | 44.8 |
| 8 | OA + FV (31.25 μg/mL) | 45.2 |
| 9 | OA + FV (62.5 μg/mL) | 48.4 |

*Data measured in different set of experiments

The recorded reduction in mean fluorescence intensity values between the sets of experiments given in Table 3 and Table 4 show some variation, as expected.

A further series of experiments were performed using Rubus idaeus (RI) and Fragaria vesca (FV) extracts in combination with oleanolic acid (A), compared with other plant extracts, including Hedera helix (HH), Ilex aquifolium (IA), and Garcinia mangostana (GM), alone and together with Oleanolic Acid. The activity is also compared with epigallocatechin gallate (EGCG) and salicylic acid (SA). The results are shown in Table 5, and FIGS. 4 and 5.

In these experiments, a test composition was added to the well of a 24 well plate to achieve a final concentration of 10 μM for the compound (4 μL of a 10 mM stock solution made up in DMSO into 396 μL of culture media) or 31.25 μg/mL for the plant extract (4 μL of a 3,125 μg/mL stock solution made up in DMSO into 396 μL of culture media).

TABLE 5
In-vitro lipid reduction percentages of the entries.

| | | Percentage Reduction in Mean |
|---|---|---|
| Entry | Composition | Fluorescence Intensity vs DMSO |
| 1 | DMSO (vehicle) | 00.0 |
| 2 | EGCG | 13.4 |
| 3 | Salicylic Acid (SA) | 15.5 |
| 4 | Oleanolic Acid (OA) | 33.5 |
| 5 | Rubus idaeus (RI) | 15.7 |
| 6 | Hedera helix (HH) | 0.8 |
| 7 | Ilex aquifolium (IA) | 0.3 |
| 8 | Garcinia Mangostana (GM) | 30.0 |
| 9 | Fragaria vesca (FV) | 12.2 |
| 10 | OA + RI | 41.7 |
| 11 | OA + HH | 41.8 |
| 12 | OA + IA | 38.9 |
| 13 | OA + GM | 42.4 |
| 14 | OA + FV | 45.2 |

The results demonstrate that a composition comprising Oleanolic Acid (OA) and a plant extract (Rubus idaeus extract (RI) and Fragaria vesca extract (FV)) provides improved lipid reduction in comparison to the individual compound or plant extract. The results demonstrate a synergist effect for the composition against the individual compound and individual extract.

For reference, data is also provided for other extracts and combinations.

### REFERENCES

A number of publications are cited above in order to more fully describe and disclose the invention and the state of the art to which the invention pertains. Full citations for these references are provided below. The entirety of each of these references is incorporated herein.
Cheng et al., “Sebocytes, multifaceted epithelial cells: lipid production and holocrine secretion”, Int. J. Biochem. Cell Biol., 2010; Vol. 42, pp. 181-185.
WO 2020/263188

1. A composition comprising:
oleanolic acid and the salts and solvates thereof; and
one or more compounds selected from the group consisting of ellagic acid, adenine, betaine, choline, miquelianin, p-coumaric acid, pipecolic acid, protocatechuic acid and tyramine, and the salts, prodrugs and solvates thereof.

2. The composition of claim 1, wherein the one or more compounds are selected from the group consisting of ellagic acid, adenine, betaine, choline, miquelianin and tyramine, and the salts, prodrugs and solvates thereof.

3. The composition of claim 1, wherein the one or more compounds are selected from the group consisting of ellagic acid, betaine, choline, miquelianin and tyramine, and the salts, prodrugs and solvates thereof.

4. The composition of claim 1, comprising two or more compounds selected from the group, such as three or more compounds, such as where at least one compound is ellagic acid.

5. The composition of claim 4, comprising:
oleanolic acid and the salts and solvates thereof; and
a combination of ellagic acid, adenine, betaine, choline, miquelianin, p coumaric acid, pipecolic acid and tyramine.

6. The composition of claim 4, comprising:
oleanolic acid and the salts and solvates thereof; and
a combination of ellagic acid, adenine, betaine, choline, miquelianin, p coumaric acid, and pipecolic acid.

7. The composition of claim 1, wherein the mole ratio of oleanolic acid to a compound selected from the group is 1:x, where x is in the range 0.1 to 20.

8. The composition of claim 7, wherein the mole ratio of oleanolic acid to a compound selected from the group is 1:x, where x is in the range 1 to 10, such as 2 to 10, such as 5 to 10, such as about 8.

9. The composition of claim 1, comprising:
oleanolic acid and the salts and solvates thereof; and
a plant extract comprising the one or more compounds.

10. The composition of claim 9, wherein the plant extract is a Fragaria vesca extract, a Rubus idaeus extract, or a Fragaria×ananassa extract, such as a Fragaria vesca extract or a Rubus idaeus extract.

11. A skin care product selected from skin/hand lotion, skin/hand cream, skin/hand ointment, skin/hand paste, skin toner, shaving gel, shaving cream, sunscreen, deodorant, antiperspirant, suntan lotion, after sun, aftershave, body oil, bath oil and bubble bath, the skin care product comprising the composition of claim 1.

12. A hair care product selected from conditioner, hair detangling lotion, styling gel, styling cream, styling wax, styling lotion, mousse, spray gel, hair tonic, spritz and pomade, the hair care product comprising the composition of claim 1.

13. A cosmetic method for decreasing lipid production in the skin of an individual, the method comprising contacting the skin with the composition of claim 1.

14. A method for decreasing lipid production in sebocytes or in the skin of an individual, the method comprising contacting the skin with the composition of claim 1, wherein the method is not a method of treatment.

15. A method for decreasing lipid production in sebocytes, the method comprising contacting the sebocytes with the composition of claim 1.

16. The composition of claim 1 for use in decreasing lipid production in sebocytes.

17. The composition of claim 1 for use in a method of treatment.

18. The composition of claim 1 for use in a method of treating a skin disease or disorder associated with over-production of lipids in the skin.

19. The composition for use of claim 18, wherein the disease or disorder is selected from acne vulgaris and rosacea.

20. Use of the composition of claim 1 for decreasing lipid production in sebocytes.
